(19) United States
(12) Patent Application Publication
(10) Pub. No.: US 2023/0028814 A1
(43) Pub. Date: Jan. 26, 2023

(54) METHOD FOR DELIVERING A CHEMISTRY TO MICROCHAMBERS VIA A CHANNEL

(71) Applicant: MOUND LASER & PHOTONICS CENTER, INC., Nashua, NH (US)

(72) Inventors: Terry Rusch, Vadnais Heights, MN (US); Alex Jennings, New Richmond, WI (US); Scott Johansen, Saint Paul, MN (US)

(21) Appl. No.: 17/872,724

(22) Filed: Jul. 25, 2022

Related U.S. Application Data
(60) Provisional application No. 63/225,768, filed on Jul. 26, 2021.

(51) Int. Cl. C12M 3/06; B01L 3/00

(52) U.S. Cl. CPC C12M 23/16; B01L 3/5027

(57) ABSTRACT

A method for filling microchambers with a chemistry in a substrate containing a plurality of microchambers comprises forming a channel in the substrate such that the channel is fluidically connected with the plurality of microchambers. The chemistry is delivered into the channel so that the chemistry is delivered to each of the microchambers. The chemistry is then permitted to incubate within each of the microchambers. Excess chemistry is then removed from the microchambers by introducing fluid through the channel to each of the connected microchambers.

### CROSS REFERENCE TO RELATED APPLICATION

This application claims the priority benefit of U.S. provisional application Ser. No. 63/225,768 filed Jul. 26, 2021. The aforementioned application is incorporated herein by reference in its entirety.

### BACKGROUND

This disclosure relates to the formation of microfluidic channels through the use of a laser, and in particular, the formation of microfluidics channels in thin substrates to disperse a chemistry to areas of interest.

Microfluidics relates to the science and the technique for flow management at the micro-scale. At the micro-scale, fluid dynamics radically change when compared to macro-fluidic streams. For purposes of this patent application, the term microfluidics will refer to channels having a cross sectional area of 1 millimeter square (1 mm²) or less. In certain embodiments, the height of such microfluidic channels is less than 500 μm and have a well volume of 3 microliters (3 μL) or less. At the micro scale, fluids positioned within microchannels (for example channel size of around 100 nanometers to 500 micrometers) may differ from “macro-fluidic” behavior in that factors such as surface tension, energy dissipation, and fluidic resistance start to dominate. At the micro scale, fluid streams tend to be laminar (have a low Reynolds number), that is, they do not mix or form vortexes or eddies. Turbulent flow does not exist within a micro-channel.

Microfluidic channels are generally produced within chips, commonly referred to as microfluidic chips. Microfluidic chips are used to carefully manage fluids and integrate several functions that generally would require an entire laboratory. Often microfluidic chips are referred to as a “laboratory-on-chip.”

In the past, several materials have been used to create microfluidic chips, materials such as glass, silicon and polymers. Polymers have become the material of choice for such chips due to their transparency, elasticity, cost, and permeability in some situations.

In addition to the above attributes, for a polymer to be used as a microfluidic chip, microfluidic channels allow for a reduction in many situations of a required sample and reagent volume. In many situations, the amount of the required sample (obtained from a patient) is limited in the case of neonatal diagnostics or analysis of proteins. With respect to reagent volume, many of the reagents used, for example to identify and analyze a protein, are very expensive. Thus, miniaturization has significant cost advantages in addition to reduction in the amount of time to provide an answer to the patient or clinician.

### SUMMARY

This disclosure relates to a method for filling microchambers with a chemistry in a substrate containing a plurality of microchambers. The method comprises forming a channel in the substrate such that the channel is fluidically connected with the plurality of microchambers. The chemistry is delivered into the channel so that the chemistry is delivered to each of the microchambers. The chemistry is then permitted to incubate within each of the microchambers. Excess chemistry is then removed from the microchambers by introducing fluid through the channel to each of the connected microchambers.

In certain embodiments, the channel is a microchannel. In a more limited aspect, the microchannel has a cross sectional area of 1 mm² or less. In another more limited aspect, the microchannel has a volume of 3 microliters or less. In another more limited aspect, the microchannel has a height of less than 500 μm. In another more limited aspect, the microchannel is covered by a masking layer.

In certain embodiments, the steps of delivering the chemistry, permitting the chemistry to incubate, and removing excess chemistry are repeated until a selected number of chemistries are disposed within the microchambers. In a more limited aspect, the channel is a microchannel. In another more limited aspect, the channel is covered by a masking layer.

In certain embodiments, the channel is covered by a masking layer.

In certain embodiments, the microchambers are connected in series by the channel.

In certain embodiments, the microchambers are connected in parallel to the channel.

### BRIEF DESCRIPTION OF THE DRAWINGS

The invention may take form in various components and arrangements of components, and in various steps and arrangements of steps. The drawings are only for purposes of illustrating preferred embodiments and are not to be construed as limiting the invention.

FIG. 1 is a photographic view of a channel being used to deliver a chemistry to a plurality of microchambers.

FIG. 2 is a photographic view of a plurality of microchannel sections being used to deliver a chemistry to a plurality of microchambers.

### DETAILED DESCRIPTION

Reference will now be made in detail to presently preferred embodiments, one or more examples of which are illustrated in the accompanying drawings. Each example is provided by way of explanation of the invention, not limitation of the invention.

The terms “a” or “an,” as used herein, are defined as one or more than one. The term “another,” as used herein, is defined as at least a second or more. The terms “including” and/or “having” as used herein, are defined as “comprising” (i.e., open transition). Unless specifically stated otherwise, the terms “attached,” “coupled,” “operatively coupled,” “joined”, and the like are defined as indirectly or directly connected.

As used in this application, the terms “front,” “rear,” “upper,” “lower,” “upwardly,” “downwardly,” “left,” “right,” and other orientation descriptors are intended to facilitate the description of the exemplary embodiment(s) of the present invention, and are not intended to limit the structure thereof to any particular position or orientation.

All numbers herein are assumed to be modified by the term “about,” unless stated otherwise. The recitation of numerical ranges by endpoints includes all numbers subsumed within that range (e.g., 1 to 5 includes 1, 1.5, 2, 2.75, 3, 3.80, 4, and 5).

In one particular application, a microfluidic chip may contain several microchambers, each having a chemistry for a specific identification or use. For purposes of this application, the words chemistry or chemistries mean one or more chemical or biological components, or a combination of both, which are to be deposited and then contained in an area or a microchamber of a microfluidic chip for subsequent use. Such chemical or biological components may also be referred to as reagents. The chemistries described herein may find applications which include but are not limited to microfluidic Point of Care (POC) testing and Advanced Wound Care (AWC) applications. In the past, in order for the chemistry in the microfluidic chambers to function properly, a mask is used in the application to capture the chemistry in a specific area or chamber. There are inherent issues with delivering such chemistries using this format. The issues include cross-contamination, missed dispensing of the chemistry, slow speed in delivering the chemistry to the microchambers, spraying/splattering of the chemistry, and then the difficulty of coating complex geometries. Other issues in specific applications include incubation steps to prevent evaporation of the chemistry from the microchambers.

The word microchamber as used here in shall mean any area of which is the destination for a chemistry. The phrase “area of interest” shall mean the same as microchamber.

This disclosure relates to delivery of a chemistry to a plurality of microchambers or areas of interest located on a chip through the use of microfluidic channels. In certain embodiments, such microfluidic channels have a cross sectional area of 1 millimeter square or less. In certain embodiments, such microfluidic channels have a cross sectional area in the range of from 0.0001 mm² to about 1 mm². In certain embodiments, such microfluidic channels have a cross sectional area in the range of from 0.001 mm² to about 1 mm². In certain embodiments, such microfluidic channels have a cross sectional area in the range of from 0.01 mm² to about 1 mm². In certain embodiments, such microfluidic channels have a cross sectional area in the range of from 0.1 mm² to about 1 mm².

In certain embodiments, the height of such microfluidic channels is 500 μm or less. In certain embodiments, the height of such microfluidic channels is in the range of from 0.01 μm to 500 μm. In certain embodiments, the height of such microfluidic channels is in the range of from 0.05 μm to 500 μm. In certain embodiments, the height of such microfluidic channels is in the range of from 0.1 μm to 500 μm. In certain embodiments, the height of such microfluidic channels is in the range of from 0.5 μm to 500 μm. In certain embodiments, the height of such microfluidic channels is in the range of from 1 μm to 500 μm. In certain embodiments, the height of such microfluidic channels is in the range of from 5 μm to 500 μm. In certain embodiments, the height of such microfluidic channels is in the range of from 10 μm to 500 μm. In certain embodiments, the height of such microfluidic channels is in the range of from 50 μm to 500 μm. In certain embodiments, the height of such microfluidic channels is in the range of from 100 μm to 500 μm. In certain embodiments, the height of such microfluidic channels is in the range of from 300 μm to 500 μm. In embodiments, the height of such microfluidic channels is approximately 300 μm.

In certain embodiments, the well volume of such microfluidic channels is 3 microliters or less. In certain embodiments, the well volume of such microfluidic channels is in the range of from 0.0001 μL to 3 μL. In certain embodiments, the well volume of such microfluidic channels is in the range of from 0.0003 μL to 3 μL. In certain embodiments, the well volume of such microfluidic channels is in the range of from 0.001 μL to 3 μL. In certain embodiments, the well volume of such microfluidic channels is in the range of from 0.003 μL to 3 μL. In certain embodiments, the well volume of such microfluidic channels is in the range of from 0.01 μL to 3 μL. In certain embodiments, the well volume of such microfluidic channels is in the range of from 0.03 μL to 3 μL. In certain embodiments, the well volume of such microfluidic channels is in the range of from 0.1 μL to 3 μL. In certain embodiments, the well volume of such microfluidic channels is in the range of from 0.3 μL to 3 μL. In certain embodiments, the well volume of such microfluidic channels is in the range of from 1 μL to 3 μL.

The process disclosed herein solves the issues of cross-contamination, missed dispensing of the chemistry, increases the speed in which the chemistry is delivered to microchambers, eliminates spraying and splattering of the chemistry and provides the ability to coat complex geometries without difficulty. Furthermore, in using the process of this disclosure, the need to place the chip in a humidity-controlled environment to prevent evaporation of the chemistry may no longer be needed.

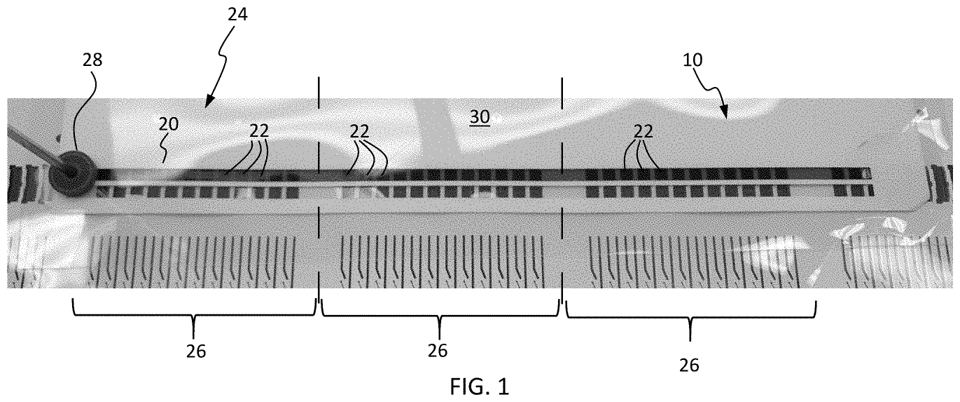

The process of this disclosure utilizes channels and, in some applications, microfluidic channels, to quickly disperse/dispense the chemistry to the area or areas of interest. Using microfluidic layers is the basis for many POC tests. In one embodiment, as illustrated in FIG. 1, a laser is used to create a channel 20 in a substrate 10 to form a test strip 24. The channel 20 is fluidly connected in parallel to a plurality of areas or microchambers 22 to which chemistry is to be sent. The microchambers 22 are positioned such that the flow of the chemistry will occur into each microchamber from the channel 20. In this particular embodiment, a number of chemistries will be coated within each area of interest 22, as explained further.

A top layer 30, which is transparent in this particular embodiment, is attached to the substrate 10 below which contains the channel 20. Below the channel 20 are disposed the areas of interest 22. In a typical application, a number of chemistries are conveyed to each area of interest 22 and permitted to develop/incubate to result in a coating. An applicator 28 provides a first chemistry which is conveyed through the channel 20 and into each area of interest 22. It is important to note that the first chemistry is deposited in each area of interest with one step of transporting the first chemistry through the channel 20. Typically, a peristaltic pump (not shown) provides the motive force to transport the chemistry. The chemistry is allowed to incubate and is then sucked out via the channel 20, thereby removing excess of the first chemistry from each area of interest 22. The areas of interest 22 are then washed with a solution via the channel 20 and the washing fluid is then removed in a similar manner to prepare all of the areas of interest 22 for the next chemistry.

In certain embodiments, a next or second chemistry is delivered to each area of interest in a manner similar to the delivery of the first chemistry to the areas of interest 22. Once the next or second chemistry is allowed to incubate and excess chemistry is removed, the areas of interest 22 are cleansed for the next chemistry. The areas of interest 22 may be coated several times each in a similar manner quickly without cross-contamination and missed dispensing of the chemistries, all of which are accomplished in a very time efficient manner.

Once all of the chemistries are deposited or coated within the areas of interest 22, the strip 24 is then cut into individual discrete cards 26 (as indicated by the broken lines) for subsequent use.

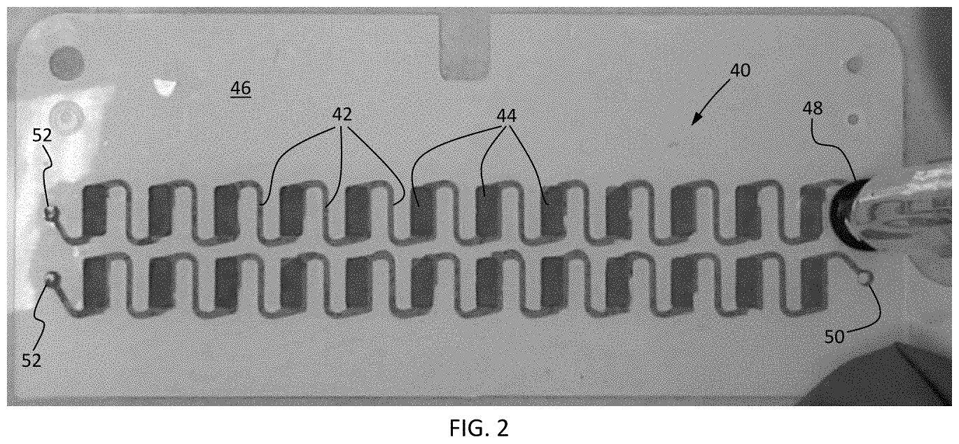

In another embodiment as illustrated in FIG. 2, a plurality of areas of interest 44 are fluidly connected by microfluidic channels 42 in a series arrangement. Both the microfluidic channels 42 and the areas of interest 44 are created in a card 40 with a mask 46 (which is transparent in the specific embodiment shown in FIG. 2 although not necessary) overlying the areas of interest 44 and the microfluidic channels 42.

Similar to the embodiment illustrated in FIG. 1, a desired chemistry is delivered to each individual area of interest 44. The area of interest 44 may be a microchamber for holding a plurality of coatings which will subsequently be, for example, part of a POC test. Filling each microchamber 44 individually, as is commonly done with a pipette, takes considerable time, may be subject to cross-contamination, dispensing the chemistry in a particular microchamber may be missed or sprayed or splattering of all the chemistry while dispensing the chemistry into the microchamber may occur. After each chemistry is permitted to incubate and excess removed, the resulting coating is then washed and prepared for the next subsequent coating. The term incubate for purposes of this application shall mean for the chemistry to develop into a desired phase and shall include within its definition curing of a chemistry which is the reaction process of a component to a chemically reacted product or to a different physical phase of the chemical component. The embodiment illustrated in FIG. 2 eliminates all these problems as will be discussed further.

As illustrated in FIG. 2, microfluidic channel sections 42 are formed by a laser beam (not shown). A plurality of areas of interest 44 are spaced from each other, preferably in a row. Each area of interest 44 is connected to an adjacent area of interest by a microfluidic channel section 42. A dispensing unit 48, typically powered by a peristaltic pump (not shown), engages an input 50. An initial chemistry is dispensed into the input 50, which then flows through each microfluidic channel section 42 into subsequently disposed areas of interest 44 until all of the areas of interest 44 are filled with the chemistry. An output 52, which is open to the atmosphere, permits the chemistry to flow easily through the microfluidic channel sections 42 and into the areas of interest 44, filling each area of interest with the chemistry. Using the process of this disclosure eliminates virtually all air pockets, filling each area of interest regardless of the geometric complexity of the area of interest 44.

Once the chemistry has had time to incubate, the excess chemistry may be washed out of the areas of interest 44 using a suitable washing fluid that is injected using the applicator 48. The washing fluid is injected into the input 50 which results in efficient washing of each area of interest 44. The fluid used to wash each area of interest 44 then exits the output 52. This prepares each area of interest to subsequently receive another chemistry. The procedure is repeated until the areas of interest have received all of the necessary chemistries.

The description above should not be construed as limiting the scope of the invention, but as merely providing illustrations to some of the presently preferred embodiments of this invention. In light of the above description and examples, various other modifications and variations will now become apparent to those skilled in the art without departing from the spirit and scope of the present invention as defined by the appended claims. Accordingly, the scope of the invention should be determined solely by the appended claims and their legal equivalents.

What is claimed is:

1. A method for filling microchambers with a chemistry in a substrate containing a plurality of microchambers, the method comprising:
forming a channel in the substrate such that the channel is fluidically connected with the plurality of microchambers;
delivering the chemistry into the channel so that the chemistry is delivered to each of the microchambers;
permitting the chemistry to incubate within each of the microchambers; and
removing excess chemistry from the microchambers by introducing fluid through the channel to each of the connected microchambers.

2. The method of claim 1, wherein the channel is a microchannel.

3. The method of claim 2, wherein the microchannel has a cross sectional area of 1 mm² or less.

4. The method of claim 2, wherein the microchannel has a volume of 3 microliters or less.

5. The method of claim 2, wherein the microchannel has a height of less than 500 μm.

6. The method of claim 2, wherein the microchannel is covered by a masking layer.

7. The method of claim 1, wherein the steps of delivering the chemistry, permitting the chemistry to incubate, and removing excess chemistry are repeated until a selected number of chemistries are disposed within the microchambers.

8. The method of claim 7, wherein the channel is covered by a masking layer.

9. The method of claim 1, wherein the channel is covered by a masking layer.

10. The method of claim 1, wherein the microchambers are connected in series by the channel.

11. The method of claim 1, wherein the microchambers are connected in parallel to the channel.
